(19) United States
(12) Patent Application Publication
(10) Pub. No.: US 2026/0144951 A1
(43) Pub. Date: May 28, 2026

(54) USE OF POSITIVE AIRWAY PRESSURE THERAPY TO TREAT GLAUCOMA

(71) Applicant: 0891771 BC LTD., Savona (CA)

(72) Inventor: David STEWART, Savona (CA)

(21) Appl. No.: 18/958,361

(22) Filed: Nov. 25, 2024

(51) Int. Cl. A61M 16/06; A61K 31/05

(52) U.S. Cl. CPC A61M 16/0616; A61K 31/05

(57) ABSTRACT

Methods of administering positive airway pressure therapy to treat glaucoma in a subject and uses of positive airway pressure therapy for treating glaucoma in a subject are provided. Methods of administering positive airway pressure therapy to treat glaucoma in a subject with sleep apnea or vascular dysregulation and uses of positive airway pressure therapy for treating glaucoma in a subject with sleep apnea or vascular dysregulation are also provided.

### TECHNICAL FIELD

This invention relates to the treatment of glaucoma, including open angle glaucoma.

### BACKGROUND

Glaucoma is a group of chronic and progressive eye diseases that can cause vision loss and blindness by damaging the optic nerve of the eye. Open angle glaucoma is the major form of glaucoma and is a clinical condition that results in progressive damage to the optic nerve head in the retina. Open angle glaucoma results, for example, in measurable thinning of the retinal layer, loss of ganglion cells and their axons, and tissue remodelling involving both the optic nerve head and the retina. Further, there are visual field defects (e.g. visual field and vision loss) and reduction in the diameter of arterioles in the retinal layer. Generally, retinal thinning and/or visual field defects are the basis for a diagnosis of glaucoma.

Open angle glaucoma is generally considered an irreversible process that leads to blindness. This process is referred to as “progression”. Open angle glaucoma is a major cause of permanent blindness in the world.

There is a general desire to treat open angle glaucoma by slowing, stopping or reversing its progression.

Open angle glaucoma has traditionally been categorized into two types primarily determined by the intraocular pressure of the eye prior to glaucoma treatment: normal tension glaucoma and high tension glaucoma. Under this approach, an intraocular pressure of 21 mmHg has been cited as the threshold separating normal tension glaucoma from high tension glaucoma. Specifically, a patient would be considered to have high tension glaucoma where the intraocular pressure is above 21 mmHg. Correspondingly, an intraocular pressure of 21 mmHg or below would classify a patient as having normal tension glaucoma.

In high tension glaucoma as defined by the intraocular pressure cut-off criterion approach, it has been considered that damage to the retina is principally due to the obstruction of aqueous outflow within the trabecular meshwork in the front of the eye, causing an increase in ocular pressure to a point where nerve damage occurs at the retina in the back of the eye. Elevated intraocular pressure exerts direct mechanical damage to the optic nerve head by restricting blood flow. Reducing intraocular pressure has been found to generally be effective in slowing progression of the disease, and therefore the standard treatment for high tension glaucoma is decreasing intraocular pressure.

Reduction in intraocular pressure can be achieved, for example, by using drugs such as prostaglandin analogues including latanoprost, applying laser energy to the trabecular network to improve fluid flow, or surgical creation of a fistula from the anterior chamber to the subconjunctival space of the eye (trabeculectomy).

By contrast, it has been considered that in normal tension glaucoma, damage to the optic nerve can occur without an increase in intraocular pressure, making normal tension glaucoma unique among glaucomas. While the mechanism and causes of normal tension glaucoma are not presently completely understood, proposed mechanisms for normal tension glaucoma include:
sensitivity in intraocular pressure such that in certain individuals, even normal intraocular pressures can lead to damage to the optic nerve;
local or generalized vascular dysregulation;
a higher than normal pressure gradient across the lamina cribrosa, which consists of a porous collagen structure through which the axons of retinal ganglion cells pass on their path from the retina to the brain; and
impaired cerebral spinal fluid circulation in the subarachnoid space of the optic nerve resulting in toxic damage to the nerve.

Notably, research has found that individuals with normal tension glaucoma are more likely to have several characteristics and/or symptoms known to be associated with vascular dysregulation compared to individuals without normal tension glaucoma.

Vascular dysregulation, also referred to as Flammer Syndrome, is one of the proposed mechanisms or contributing factors for normal tension glaucoma. Specifically, vascular dysregulation can result in variations in retinal blood flow, which may be a factor in the development of normal tension glaucoma. Individuals with vascular dysregulation also have a predisposition for an altered reaction of the blood vessels to stimuli like cold or emotional stress. Patients with vascular dysregulation have been found to have the following characteristics and/or symptoms: cold hands and/or feet, reduced feeling of thirst, low blood pressure, dizziness, increased sensitivity to certain drugs, migraines, headaches, tinnitus, low body weight, feeling cold, long sleep onset time, good smell perception, increased pain sensation, skin blotches, and/or tendency towards perfectionism.

Despite the unique characteristics of normal tension glaucoma noted above, the standard treatment for normal tension glaucoma has typically been the same as for high tension glaucoma, namely lowering of intraocular pressure with drugs such as prostaglandin analogues or by other methods. Lowering intraocular pressure has, notably, been shown to slow the progression of normal tension glaucoma in a majority of patients. However, for a significant subset of patients (at least around 20%), lowering intraocular pressure has no effect on disease progression. This suggests that classification using the intraocular pressure cut-off criterion may not be applicable and/or useful in every case and further suggests that other mechanisms, besides intraocular pressure, are involved in the progression of normal tension glaucoma.

Indeed, while the distinction between normal tension glaucoma and high tension glaucoma using the intraocular pressure cut-off criterion approach (i.e., at an intraocular pressure of 21 mmHg) has become standard in the field, it has been recently studied and discussed whether this approach is applicable with respect to structural and functional differences between pressure-defined glaucoma types. This is especially in view of the known variability in measurements of intraocular pressure and variability between groups. In this regard, at least one recent study investigating whether there were quantifiable structural or functional differences between high tension glaucoma and low tension glaucoma, as defined using the 21 mmHg cut off criterion between these types of glaucoma, found that quantitative structural or functional parameters could not distinguish between pressure-defined glaucoma types at the point of diagnosis. This study further concluded that:
there is substantial heterogeneity among structural and functional results of patients newly diagnosed with glaucoma at different intraocular pressure levels;
no quantitative clinical parameters that could meaningfully discriminate between newly diagnosed high-tension glaucoma and normal tension glaucoma patients; and
a meaningful distinction of quantitative outputs at the time of diagnosis is unlikely to be achieved due to the heterogeneity of presentation at the individual level.

Accordingly, this study suggests that at the level of an individual patient, diagnosis of normal tension glaucoma or high tension glaucoma using the standard intraocular pressure cut-off criterion approach does not meaningfully distinguish between structural and functional characteristics of a patient's glaucoma. It follows that while diagnosis of pressure-defined glaucoma types may be useful in some regards, that at the level of an individual patient this may not be the case.

There is a general desire for new treatment methods to slow or stop the progression of glaucoma, and specifically open angle glaucoma including high tension glaucoma and normal tension glaucoma. There is a general desire for new treatment methods to reverse the progression of glaucoma, and specifically open angle glaucoma including high tension glaucoma and normal tension glaucoma.

There is a general desire for treatments for glaucoma, particularly for patients for which lowering intraocular pressure has no effect on the progression of the glaucoma.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a method of treating open angle glaucoma in a subject in need thereof, the method comprising administering positive airway pressure (PAP) therapy to the subject. In some embodiments, the subject additionally suffers from sleep apnea and the PAP therapy is administered to the subject when the subject is asleep.

In some embodiments, the PAP therapy is continuous positive airway pressure (CPAP) therapy. In other embodiments, the PAP therapy is auto-adjusting positive airway pressure (APAP) therapy, bilevel PAP therapy, or adaptive servo-ventilation (ASV) therapy. In some embodiments, the PAP therapy is administered using a PAP machine. In some embodiments, the PAP machine is a CPAP machine, an APAP machine, bilevel PAP machine, or ASV machine.

In some embodiments, the subject with open angle glaucoma has an intraocular pressure of less than or equal to 21 mmHg. In some embodiments, the subject has been diagnosed with normal tension glaucoma. In other embodiments, the subject with open angle glaucoma has an intraocular pressure of greater than 21 mmHg. In some embodiments, the subject has been diagnosed with high tension glaucoma.

In some embodiments, the subject has at least one of retinal thinning, an increased cup volume, or an increased cup to disk ratio, resulting from the progression of the open angle glaucoma.

In some embodiments, the subject has initial vision loss resulting from progression of the open angle glaucoma and/or has one or more scotomas resulting from the progression of the open angle glaucoma. A scotoma is a visual field abnormality, also known as a blind spot.

In some embodiments, the sleep apnea is obstructive sleep apnea. In other embodiments, the subject has an apnea hypopnea index (AHI) of greater than or equal to 5. In further embodiments, the subject has an AHI of greater than or equal to 5 and less than or equal to 15. In other embodiments, the PAP therapy reduces the AHI of the subject to less than or equal to 5.

In some embodiments, the subject additionally suffers from vascular dysregulation or Flammer Syndrome.

In some embodiments, treating the open angle glaucoma results in reversing the progression of the open angle glaucoma. In some embodiments, reversing the progression of the open angle glaucoma comprises partially or fully reversing the retinal thinning. In other embodiments, reversing the progression of the open angle glaucoma comprises at least one of deceasing the cup volume and decreasing the cup to disk ratio. In some embodiments, reversing the progression of the open angle glaucoma comprises partially or fully reversing the initial vision loss of the subject and partially or fully restoring the subject's vision. In some embodiments, reversing the progression of the open angle glaucoma comprises reducing the size or area of at least one of the one or more scotomas and/or eliminating at least one of the one or more scotomas.

In some embodiments, treating the open angle glaucoma results in slowing or stopping the progression of the open angle glaucoma. In some embodiments, slowing the progression of the open angle glaucoma comprises slowing a rate at which the subject develops further vision loss relative to the initial vision loss resulting from the progression of the open angle glaucoma. In other embodiments, stopping the progression of the open angle glaucoma comprises stopping the subject from developing further vision loss relative to the initial vision loss resulting from the progression of the open angle glaucoma. In further embodiments, slowing the progression of the open angle glaucoma comprises slowing a rate of increase in size or area of the one or more scotomas and/or slowing development of new scotomas. In even further embodiments, stopping the progression of the open angle glaucoma comprises stopping the one or more scotomas from increasing in size or area and/or stopping the development of new scotomas.

In some embodiments, the method further comprises administering an intraocular pressure reducing drug to the subject. In some embodiments, the intraocular pressure reducing drug is a prostaglandin analogue. In some embodiments, the prostaglandin analogue is latanoprost. In some embodiments, the latanoprost is administered at 0.005%.

In some embodiments, the intraocular pressure reducing drug reduces the intraocular pressure of the subject. In some embodiments, the intraocular pressure reducing drug reduces the intraocular pressure of the subject by at least 30%.

In some embodiments, the PAP therapy is administered after an initial period of administration of the intraocular pressure reducing drug. In some embodiments, the initial period of administration of the intraocular pressure reducing drug is at least 2 months. In some embodiments, the initial period of administration of the intraocular pressure reducing drug does not slow, stop or reverse the progression of the open angle glaucoma in the subject.

In some embodiments, the PAP therapy and the intraocular pressure reducing drug exhibit a synergistic effect in treating the open angle glaucoma in the subject.

In some embodiments, the patient is diagnosed with sleep apnea after being diagnosed with open angle glaucoma. In some embodiments the patient is diagnosed with vascular dysregulation and/or Flammer syndrome after being diagnosed with open angle glaucoma.

In addition to the exemplary aspects and embodiments described above, further aspects and embodiments will become apparent by reference to the drawings and by study of the following detailed descriptions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures of the drawings. It is intended that the embodiments and figures disclosed herein are to be considered illustrative rather than restrictive.

FIG. 1 shows the results of first and second visual field tests (Humphrey automated static perimeter tests) performed at the time glaucoma was diagnosed in the patient and around four months after diagnosis.

FIG. 2 shows the results of third and fourth visual field tests (Humphrey automated static perimeter tests) performed around 10 months after and around 18 months after the earliest visual field test of FIG. 1 in the same patient.

FIG. 3 shows the results of fifth and sixth visual field tests (Humphrey automated static perimeter tests) performed around 23 months after and around 30 months after the earliest visual field test of FIG. 1 in the same patient.

FIG. 4 shows the results of seventh and eighth visual field tests (Humphrey automated static perimeter tests) performed around 36 months after and around 66 months after the earliest visual field test of FIG. 1 in the same patient.

FIG. 5 shows data from a first diagnostic report assessing retinal characteristics and retinal health performed at the same time as the eighth visual field test in FIG. 4 around 66 months after the earliest visual field test of FIG. 1 in the same patient. FIG. 5A shows a summary of the average RNFL thickness, rim area, disc area, average cup to disc ratio, vertical cup to disc ratio and cup volume. FIG. 5B shows a RNFL thickness map. FIG. 5C shows a RNFL deviation map. FIGS. 5D to 5F show an extracted horizontal tomogram, extracted vertical tomogram and RNFL circular tomogram, respectively. FIG. 5G shows a neuro-retinal rim thickness plot. FIG. 5H shows a RNFL thickness plot and FIG. 5I shows corresponding RNFL quadrants and RNFL ClockHours in relation to the RNFL thickness plot of FIG. 5H.

FIG. 6 shows the results of a ninth visual field test (Humphrey automated static perimeter tests) performed around 86 months after the earliest visual field test of FIG. 1 in the same patient.

FIG. 7 shows data from a second diagnostic report assessing retinal characteristics and health performed at the same time as the ninth visual field test of FIG. 6 around 86 months after the earliest visual field test of FIG. 1 in the same patient. FIG. 7A shows a summary of the average RNFL thickness, rim area, disc area, average cup to disc ratio, vertical cup to disc ratio and cup volume. FIG. 7B shows a RNFL thickness map. FIG. 7C shows a RNFL deviation map. FIGS. 7D to 7F show an extracted horizontal tomogram, extracted vertical tomogram and RNFL circular tomogram, respectively. FIG. 7G shows a neuro-retinal rim thickness plot. FIG. 7H shows a RNFL thickness plot and FIG. 7I shows corresponding RNFL quadrants and RNFL ClockHours in relation to the RNFL thickness plot of FIG. 7H.

FIG. 8 shows a photograph of the rear (fundus) of the eye in the same patient taken at the same time as the ninth visual field test and second diagnostic report of FIG. 7 around 86 months after the earliest visual field test of FIG. 1 in the same patient.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term “glaucoma” refers to open angle glaucoma, including glaucoma classified as normal tension glaucoma or high tension glaucoma based on intraocular pressure.

As used herein, the term “treat” refers to slowing, stopping or reversing the effects, progression and/or symptoms of a disease, disorder, or condition such as glaucoma. As used herein, the term “stopping” the effects, progression and/or symptoms of a disease, disorder, or condition such as glaucoma includes stabilizing such effects, progression and/or symptoms such that they do not continue worsening over time.

The inventor has discovered that administering positive airway pressure (PAP) therapy to a patient suffering from glaucoma can treat the patient's glaucoma by slowing, stopping or reversing the progression of the glaucoma. The inventor has discovered that administering PAP therapy to a patient suffering from glaucoma that does not respond to treatment via intraocular pressure reduction can treat the patient's glaucoma by slowing, stopping or reversing the progression of the glaucoma.

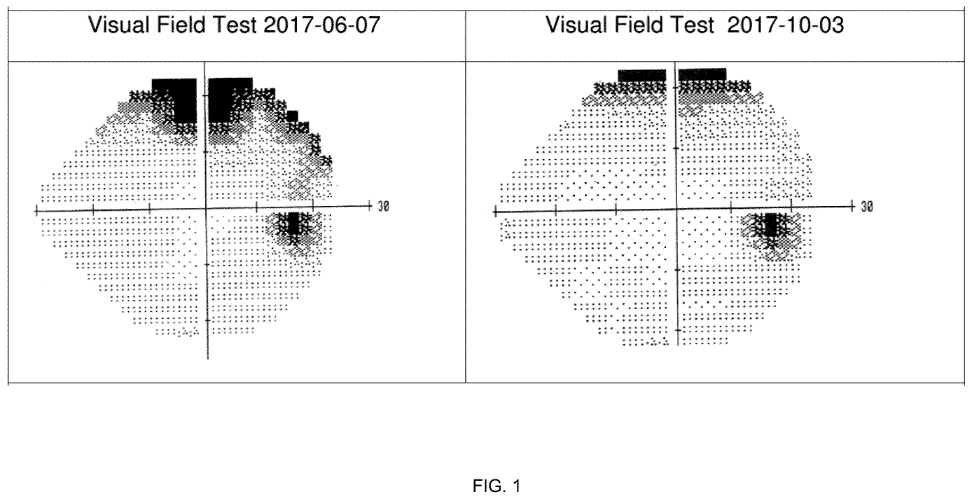

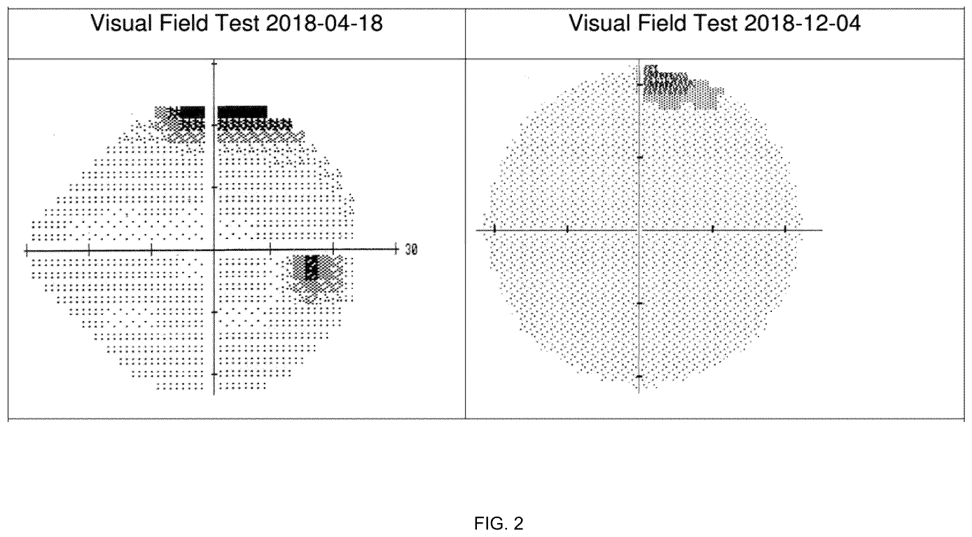

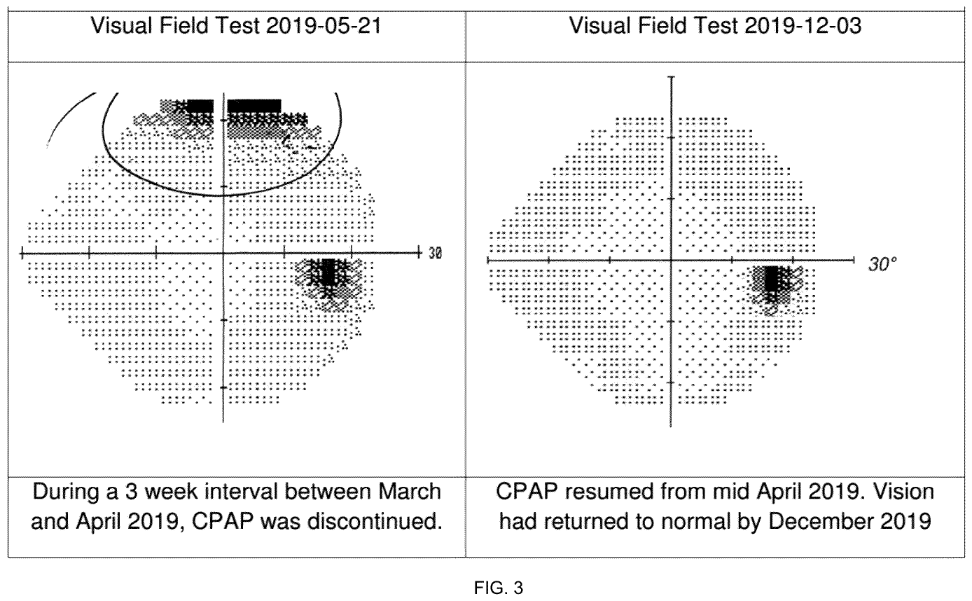

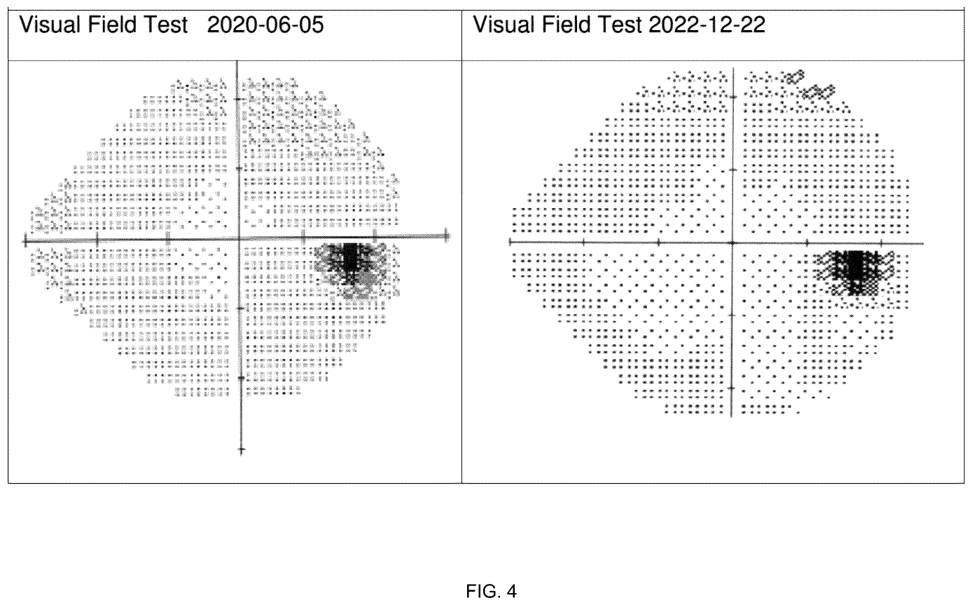

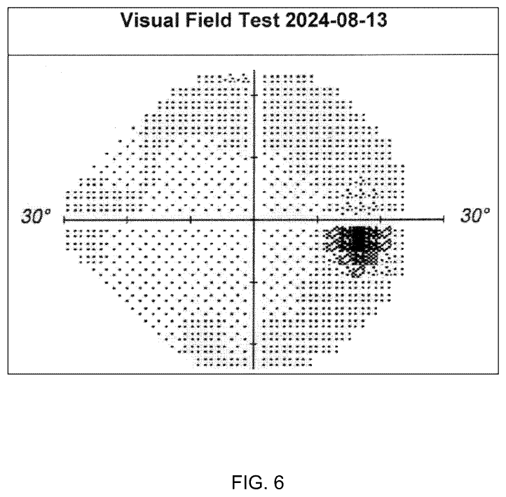

FIGS. 1-4 and 6 show the results of nine visual field tests (Humphrey automated static perimeter tests at 24-2 setting) administered to a 77 year old patient at eight different time points, the first of which was when the patient was initially diagnosed with glaucoma (FIG. 1, left panel). The second test was administered around 4 months after the first (FIG. 1, right panel). The third and fourth tests (FIG. 2, left and right panels, respectively) were administered around 10 months and 18 months after the first test, respectively. The fifth and sixth tests (FIG. 3, left and right panels, respectively) were administered around 23 months and 30 months after the first test, respectively. The seventh and eight tests (FIG. 4, left and right panels, respectively) were administered around 36 months and 66 months after the first test, respectively. The ninth test (FIG. 6) was administered around 86 months after the first test.

The Humphrey automated static perimeter test uses fixed points of light, which are shown at different intensities, over the visual field while the patient stares at a fixed point of light in the centre of the visual field and pushes a button whenever a spot of light is seen. Software then automatically varies the intensity of the light at each point to determine the intensity of light where the patient can see it 50% of the time. Dark areas in the results of these visual field tests, as seen in FIGS. 1-4, thus represent areas where a patient has a loss or partial loss of vision. In some cases (FIG. 1, both panels; FIG. 2, left panel; FIG. 3, both panels; and FIG. 4, both panels; FIG. 6), the dark area at the lower right side below the midline of the plots represent the blind spot where the nerve supply enters the retina and thus this specific dark area does not represent an area where a patient has a loss or partial loss of vision. In other cases (FIG. 2, right panel), this blind spot has been omitted by the machine used to administer the visual field test. With reference to FIGS. 1-4 and 6, the 77 year old male patient with only one eye (the right eye) was diagnosed with glaucoma in June 2017 as detected by retinal thinning and the visual appearance of the retina as shown by slit screen microscopy, and confirmed by a visual field test showing a blind spot or area of partial loss of vision (Humphrey automated static perimeter test at 24-2 setting) (FIG. 1, left panel). This blind spot or area of partial loss of vision corresponded with the areas of retinal thinning.

In this case, the patient had an intraocular pressure of 19 mmHg and was formally diagnosed with normal tension glaucoma using the intraocular pressure cut-off approach. It should also be noted that in this case, the patient's intraocular pressure of 19 mmHg is close to the intraocular pressure cut-off threshold of 21 mmHg using this approach. Thus, while the patient was formally diagnosed with normal tension glaucoma at the time of diagnosis, in view of the current understanding in the field, it would be appreciated that the patient could have been classified as either having high tension glaucoma or normal tension glaucoma, and that pressure-defined classification may not correspond to structural and functional characteristics of the patient's glaucoma. Specifically, as discussed above, there is known variability in measurements of intraocular pressure and variability between groups, and further that at the level of an individual patient, diagnosis of normal tension glaucoma or high tension glaucoma using the standard intraocular pressure cut-off criterion approach does not meaningfully distinguish between structural and functional aspects of a patient's glaucoma.

In the first visual field test shown in the left panel of FIG. 1 it can be seen from the dark area near the top center of the plot that the patient has a blind spot or area of partial loss of vision in what is otherwise a normal visual field. This dark spot or area of partial loss of vision is known as a scotoma and indicates retinal damage.

Latanoprost (0.005%) eye-drops, a prostaglandin analogue and intraocular pressure lowering medication, was prescribed to the patient immediately after the diagnosis of glaucoma (i.e., after the first visual field test shown in the left panel of FIG. 1 was performed). The latanoprost drops were administered in the right eye daily every evening from that time for at least the next 7 years and 2 months (86 months). The patient's initial intraocular pressure was 19 mmHg and the medication reduced the pressure by more than 30% as measured in the next and subsequent examinations. The eye pressure was maintained in that range over at least the next 7 years and 2 months (86 months) through the use of the latanoprost eye drops.

Subsequent second, third and fourth visual field tests indicated that the scotoma shown in the left panel of FIG. 1 had significantly decreased in size over the 18 months following the initial diagnosis of glaucoma (FIG. 1, right panel; and FIG. 2, left and right panels). As noted above, the visual field test of the right panel of FIG. 2 was done with a machine that automatically omits the blind spot where the nerve supply enters the retina, which is why this blind spot is not present in this panel. The results of the visual field tests in FIG. 1 (right panel) and FIG. 2 (left and right panels) are unexpected because they are the reverse of what would be expected to occur as glaucoma would be expected only to progress and worsen over time, even if progression was slowed by the administration of latanoprost eye drops.

Further subsequent fifth and sixth visual field tests indicated that the scotoma had returned (left panel of FIG. 3) and then subsequently disappeared (right panel of FIG. 3), respectively. Seventh and eighth visual field tests indicated that the disappearance of the scotoma seen in the right panel of FIG. 3 was maintained stably over time and the scotoma had not reappeared (FIG. 4, left and right panels).

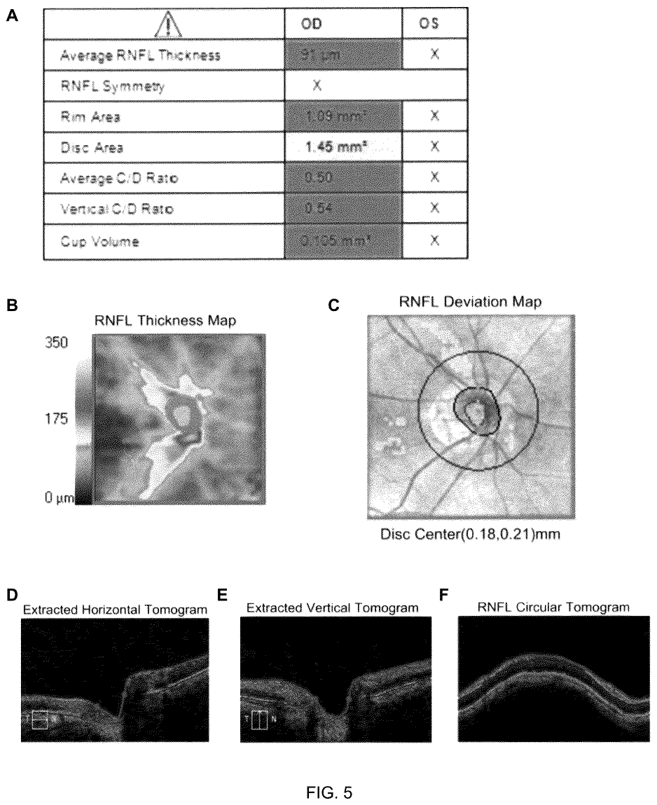

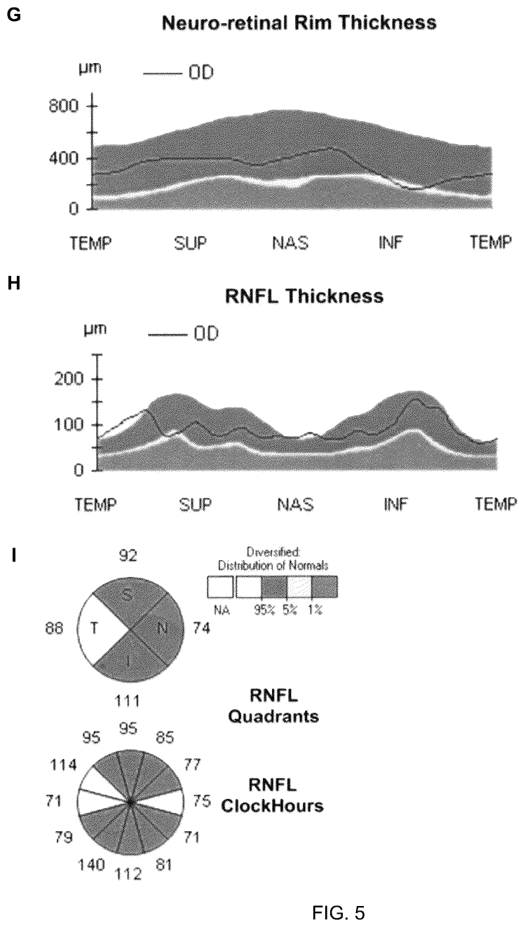

Diagnostic data regarding other parameters of the patient's eye, and particularly the retina, was gathered at the same time as the eight visual field test (right panel of FIG. 4) as seen in FIGS. 5A to 5H. This additional diagnostic data was obtained using a Zeiss™ Cirrus™ HD-OCT instrument that allows the measurement and determination of several parameters of the retina to assess retinal characteristics and health including average retinal nerve fiber layer (RNFL) thickness, rim area, disc area, cup to disc ratio, vertical cup to disc ratio and cup volume. FIGS. 5A to 5H show different data generated from the Cirrus™ instrument. Each of these figures show no evidence of retinal thinning and no evidence of glaucoma, which support and confirm the findings in the visual field tests of FIG. 3 (right panel) and FIG. 4 (left and right panels). For example, the black line in each of FIGS. 5G and 5H show the expected normal average Neuro-retinal Rim Thickness and RNFL Thickness, respectively. The Neuro-retinal Rim Thickness and RNFL Thickness measured by the Cirrus™ instrument closely match the expected average thickness measurements and indicate that the patient had a Neuro-retinal Rim Thickness and a RNFL Thickness in a healthy and/or normal range.

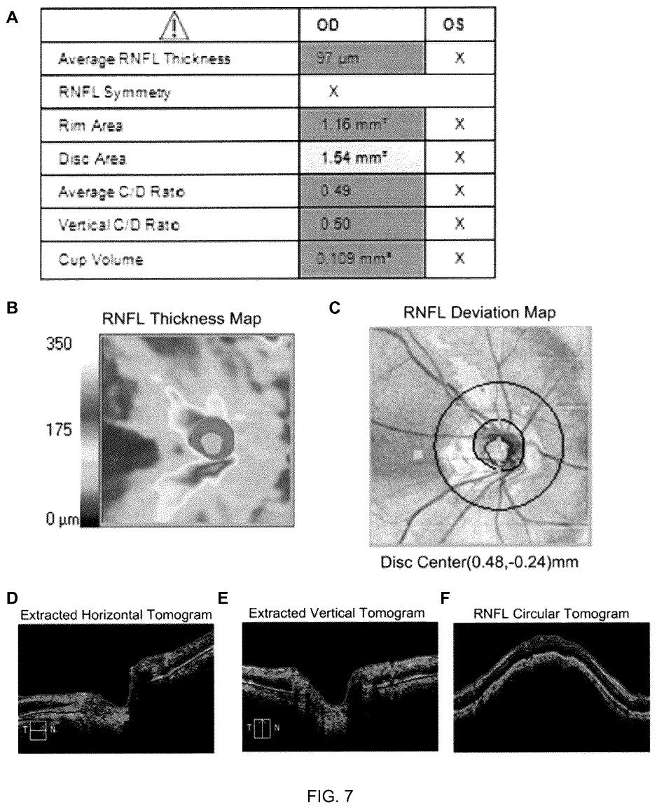

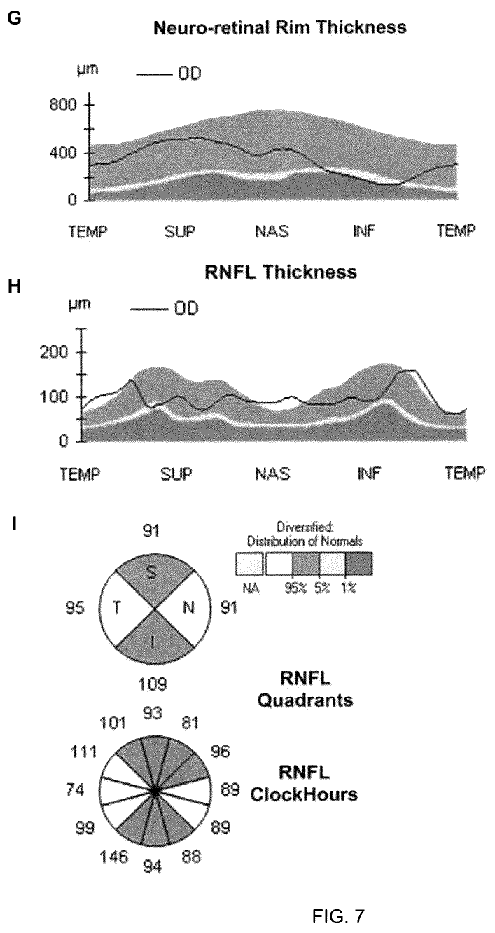

A further ninth visual field test indicated again that that the disappearance of the scotoma seen in the right panel of FIG. 3 was maintained stably over time and that the scotoma had not reappeared (FIG. 6). Diagnostic data regarding other parameters of the patient's eye, and particularly the retina, was gathered at the same time as the ninth visual field test (FIG. 6) as seen in FIGS. 7A to 7H. This additional diagnostic data was again obtained using the Cirrus™ instrument. FIGS. 7A to 7H show different data generated from the Cirrus™ instrument. Each of these figures show no evidence of retinal thinning and no evidence of glaucoma, which further support the findings in the visual field test of FIG. 3 (right panel), FIG. 4 (left and right panels) and FIG. 6. For example, the black line in each of FIGS. 7G and 7H show the expected normal average Neuro-retinal Rim Thickness and RNFL Thickness, respectively. The Neuro-retinal Rim Thickness and RNFL Thickness measured by the Cirrus™ instrument closely match the expected average thickness measurements and indicate that the patient had a Neuro-retinal Rim Thickness and a RNFL Thickness in a healthy and/or normal range.

In view of the above, and in particular FIGS. 5A to 5H and FIGS. 7A to 7H, the retinal thickness measurements from the Cirrus™ instrument show there was no detectible change in retinal thickness over at least the 20 month period between the eighth visual field test (FIG. 4) and the ninth visual field test (FIG. 6).

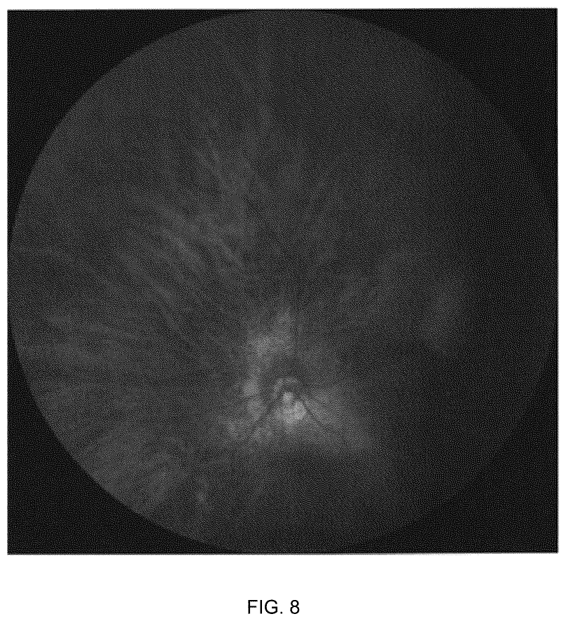

Additionally, a photograph of the rear (fundus) of the eye was taken at the same time as the ninth visual field test (FIG. 6) and second diagnostic Cirrus™ report (FIG. 8) showing that the rear of the patient's eye appears healthy and does not show any signs of glaucoma or other apparent abnormalities.

Taken together, the results of the visual fields tests and the diagnostic data using the Cirrus™ instrument indicate from the period between the fifth visual field test of FIG. 3 (left panel) and the ninth visual field test (FIG. 6), the patient had experienced a reversal of glaucoma progression (e.g., the disappearance of the scotoma and vision loss) and that the reversal of the progression of the glaucoma was stable over time (e.g., the disappearance of the scotoma and vision loss, and the lack of retinal thinning and evidence of glaucoma).

The unexpected reduction in size and gradual disappearance of the scotoma beginning with FIG. 1 (right panel) and progressing through subsequent visual fields tests as seen in FIG. 2 (left and right panels) coincided with the patient beginning treatment for obstructive sleep apnea with positive airway pressure (PAP) therapy approximately two months after the patient's diagnosis of glaucoma. Specifically, the patient began continuous positive airway pressure (CPAP) therapy using a CPAP machine in August 2017, around two months after the initial diagnosis of glaucoma. The patient continued CPAP therapy using a CPAP machine over a period of at least 7 years with the exception of a three week period. The fifth visual field test as shown in the left panel of FIG. 3, which shows the return of a scotoma, was performed shortly after this three week period without CPAP therapy. The sixth visual field test as shown in the right panel of FIG. 3 was performed after CPAP therapy was resumed following the three week period without CPAP therapy and shows the disappearance of the scotoma seen in the left panel of FIG. 3 that had returned subsequent to the three week period without CPAP therapy. Subsequent seventh and eight visual field tests, as seen in FIG. 4, did not show any scotomas or other visual field defects. Diagnostic data obtained at the same time as the eight visual field test (FIGS. 5A to 5H) indicated that there was no retinal thinning and no evidence of glaucoma. A subsequent ninth visual field test, seen in FIG. 6, did not show any scotomas and diagnostic data obtained at the same time as this ninth visual field test (FIGS. 7A to 7H) indicated that there was no retinal thinning and no evidence of glaucoma.

Taken together, these results indicate that the administration of PAP therapy (CPAP therapy using a CPAP machine) surprisingly and unexpectedly resulted in the reduction and eventual disappearance of the scotoma and the reversal in the progression of the patient's glaucoma. This is because the disappearance of the scotoma and the reversal in progression of the glaucoma remained stable over time except where PAP therapy was temporarily halted, which resulted in the scotoma returning. After resumption of the PAP therapy, the scotoma once again reduced in size/area, disappeared and did not return.

As noted previously, the patient started using latanoprost eyedrops immediately after the diagnosis of glaucoma was made and continued using the latanoprost eyedrops throughout the relevant period of 86 months including during the three week period where PAP therapy was temporarily halted. As also noted previously, the latanoprost eyedrops reduced the patient's intraocular pressure by more than 30%. Therefore, for the patient, reduction in intraocular pressure alone did not have an effect on the progression of glaucoma.

Obstructive sleep apnea is a type of sleep apnea and is a sleep disorder where recurrent episodes of complete or partial obstruction of the upper airway leads to reduced or absent breathing during sleep. These episodes are termed “apneas” with complete or near-complete cessation of breathing, or “hypopneas” when the reduction in breathing is partial. Apneas and hypopneas thus result in episodic reductions in oxygen level/saturation and can result in hypoxia.

Sleep apnea is commonly measured by a patient's apnea hypopnea index (AHI), which is the number of apneas and/or hypopneas per hour of sleep. The normal range is an AHI of 2-5. AHI can be measured using oximetry tests. An AHI of greater than 5 and less than or equal to 15 is generally considered mild sleep apnea.

PAP therapy is a non-invasive mechanical ventilation therapy where a PAP machine delivers pressurized air to open the airways of sleepers such as those with obstructive sleep apnea. The application of positive pressure prevents upper airway collapse, as occurs in obstructive sleep apnea, and thus PAP therapy is a common treatment for sleep apnea. There are several types of PAP therapy. CPAP is a type of PAP ventilation/therapy in which a constant level of pressure greater than atmospheric pressure is continuously applied using a CPAP machine to the upper respiratory tract of a patient and is commonly used to treat obstructive sleep apnea. Other types of PAP therapy that are commonly used to treat obstructive sleep apnea include, but are not limited to, auto-adjusting PAP (APAP) therapy, bilevel PAP therapy and adaptive servo-ventilation (ASV) therapy. It will be appreciated by those skilled in the art that there are several types and models of PAP machines that can administer the various types of PAP therapy to a subject. For example, CPAP therapy is administered using a CPAP machine, examples of which include the Philips Dream Machine™ and Resmed Airsense 11™.

With reference to FIGS. 1-7, the patient in this case had obstructive sleep apnea and an AHI index of 8-10, as measured by repeated oximetry tests. Oximetry on this patient showed oxygen desaturation events resulting in oxygen desaturation to 80% of normal during sleep, prior to PAP therapy. With PAP therapy (CPAP therapy using a CPAP machine), the AHI was reduced to below 2 for this patient. In this case, the patient was administered CPAP therapy using Philips Dream Machine™ and Resmed Airsense 11™ CPAP machines.

The patient in this case also had several characteristics and/or symptoms of vascular dysregulation, including cold hands and/or feet, reduced feeling of thirst, low blood pressure, migraines, tinnitus, low body weight, feeling cold, long sleep onset time and tendency towards perfectionism. While the patient was not formally diagnosed with vascular dysregulation, vascular dysregulation could explain the sensitivity of the patient to hypoxia resulting from sleep apnea.

Without being bound to any specific theory, the inventors propose that the observed reversal of glaucoma with PAP therapy, CPAP therapy in this case, may be attributed to the mitigation of hypoxic damage. Since a possible mechanism underlying normal tension glaucoma is local or generalized vascular dysregulation, temporary declines in retinal blood flow during sleep, combined with oxygen desaturation events, lead to hypoxic damage. This may be particularly pertinent in patients with vascular dysregulation. Further, it has been established that the diameter of the arteries and veins that supply blood to the retina is lower in patients with glaucoma, but that stimulation by light causes these blood vessels to increase in diameter to a level similar to non-glaucoma patients. During sleep, however, there would be no light stimulation and the lower basal rate of blood delivery to the retina would make the retina more sensitive to anoxic events. Furthermore, it is well established that hypoxia from sleep apnea can cause damage to both white and grey matter in the brain, resulting in the loss of mental function, which can be reversed by PAP therapy. Given that the retina is an extension of the brain, it is proposed that PAP therapy may similarly reverse retinal damage caused normal tension glaucoma.

Without being bound to any specific theory, the inventors propose that a patient suffering from glaucoma along with sleep apnea and/or vascular dysregulation may have glaucoma that can be stabilized or reversed with PAP therapy, and that additionally testing for sleep apnea and/or vascular dysregulation when or shortly after glaucoma is diagnosed may provide better patient treatment outcomes for glaucoma than if only intraocular pressure is considered. Current practice is focused on reduction of intraocular pressure, which is considered the gold standard treatment for glaucoma. Using intraocular pressure reduction as the only treatment, however, only slows the progress of the disease until the end point, blindness, is reached. Vascular dysregulation and sleep apnea are two variables that are not currently taken into consideration when a patient is diagnosed with glaucoma. In vascular dysregulation, a reduction of the blood supply to the retina at night results in hypoxic damage to the retina. Sleep apnea occurs more frequently with age, as is the case with glaucoma. Sleep apnea can damage many organs including the brain. The use of PAP therapy can reverse brain damage caused by sleep apnea. It is proposed that as soon as glaucoma is detected, tests should be done to detect sleep apnea and vascular dysregulation.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

1. Zhang, N., Wang, J., Li, Y., and Jiang, B. (2021). Prevalence of primary open angle glaucoma in the last 20 years: a meta-analysis and systematic review. Scientific reports, 11(1): 13762.
2. Killer, H. E. and Pircher, A. (2018). Normal tension glaucoma: review of current understanding and mechanisms of the pathogenesis. Eye, 32(5): 924-930.
3. Flammer, J. and Konieczka, K. (2017). The discovery of the Flammer syndrome: a historical and personal perspective. EPMA Journal, 8:75-97.
4. Flammer, J., Konieczka, K., and Flammer, A. J. (2013). The primary vascular dysregulation syndrome: implications for eye diseases. EPMA Journal, 4:1-33.
5. Konieczka, K., Choi, H. J., Koch, S., Fankhauser, F., Schoetzau, A., and Kim, D. M. (2017). Relationship between normal tension glaucoma and Flammer syndrome. EPMA Journal, 8:111-117.
6. Collaborative Normal-Tension Glaucoma Study Group. (1998). The effectiveness of intraocular pressure reduction in the treatment of normal-tension glaucoma. American journal of ophthalmology, 126(4): 498-505.
7. Rafla, D., Khuu, S. K., Kashyap, S., Kalloniatis, M., and Phu, J. (2023). Visualising structural and functional characteristics distinguishing between newly diagnosed high-tension and low-tension glaucoma patients. Ophthalmic and Physiological Optics, 43(4): 771-787.
8. Zhou, W. and Sabel, B. A. (2023). Vascular dysregulation in glaucoma: retinal vasoconstriction and normal neurovascular coupling in altitudinal visual field defects. EPMA Journal, 14 (1): 87-99.
9. Castravona V, Scifo P, Castellano A, Allola A, Iadanza A, Marelli S, Cappa S F, Strambi, L F, Falini A, (2014), White matter integrity in obstructive sleep apnea before and after treatment. Sleep, Vol. 37, No. 9, pp 1465-1475.

1. A method of treating open angle glaucoma in a subject in need thereof, the method comprising administering positive airway pressure (PAP) therapy to the subject, wherein:
the subject additionally suffers from sleep apnea; and
the PAP therapy is administered to the subject when the subject is asleep.

2. The method of claim 1, wherein the PAP therapy is continuous positive airway pressure (CPAP) therapy, wherein the PAP therapy is auto-adjusting positive airway pressure (APAP) therapy, bilevel PAP therapy, or adaptive servo-ventilation (ASV) therapy, wherein the PAP therapy is administered using a CPAP machine, an APAP machine, bilevel PAP machine, or ASV machine.

3. The method of claim 1, wherein the subject has an intraocular pressure of less than or equal to 21 mmHg and/or wherein the subject has been diagnosed with normal tension glaucoma.

4. The method of claim 1, wherein the subject has an intraocular pressure of greater than 21 mmHg and/or wherein the subject has been diagnosed with high tension glaucoma.

5. The method of claim 1, wherein the subject has at least one of retinal thinning, an increased cup volume, or an increased cup to disk ratio, resulting from the progression of the open angle glaucoma.

6. The method of claim 1, wherein the subject has initial vision loss resulting from progression of the open angle glaucoma and/or has one or more scotomas resulting from the progression of the open angle glaucoma.

7. The method of claim 1, wherein the sleep apnea is obstructive sleep apnea.

8. The method of claim 1, wherein the subject has an apnea hypopnea index (AHI) of greater than or equal to 5, or greater than or equal to 5 and less than or equal to 15, wherein the CPAP therapy reduces the AHI of the subject to less than or equal to 5.

9. The method of claim 1, wherein the subject additionally suffers from vascular dysregulation or Flammer Syndrome.

10. The method of claim 1, wherein treating the open angle glaucoma results in reversing the progression of the open angle glaucoma, comprising: partially or fully reversing the retinal thinning; at least one of deceasing the cup volume and decreasing the cup to disk ratio; partially or fully reversing the initial vision loss of the subject and partially or fully restoring the subject's vision; and/or reducing the size or area of at least one of the one or more scotomas and/or eliminating at least one of the one or more scotomas.

11. The method of claim 1, wherein treating the open angle glaucoma results in slowing or stopping the progression of the open angle glaucoma, comprising: slowing a rate at which the subject develops further vision loss relative to the initial vision loss resulting from the progression of the open angle glaucoma; stopping the progression of the open angle glaucoma comprises stopping the subject from developing further vision loss relative to the initial vision loss resulting from the progression of the open angle glaucoma; slowing the progression of the open angle glaucoma comprises slowing a rate of increase in size or area of the one or more scotomas and/or slowing development of new scotomas; and/or stopping the one or more scotomas from increasing in size or area and/or stopping the development of new scotomas.

12. The method of claim 1, further comprising administering an intraocular pressure reducing drug to the subject.

13. The method of claim 12, wherein the intraocular pressure reducing drug is a prostaglandin analogue, wherein the prostaglandin analogue is latanoprost, wherein the latanoprost is administered at 0.005%.

14. The method of claim 12, wherein the intraocular pressure reducing drug reduces the intraocular pressure of the subject by at least 30%.

15. The method of claim 12, wherein the CPAP therapy is administered after an initial period of administration of the intraocular pressure reducing drug.

16. The method of claim 15, wherein the initial period of administration of the intraocular pressure reducing drug is at least 2 months.

17. The method of claim 15, wherein the initial period of administration of the intraocular pressure reducing drug does not slow, stop or reverse the progression of the open angle glaucoma in the subject.

18. The method of claim 12, wherein the CPAP therapy and the intraocular pressure reducing drug exhibit a synergistic effect in treating the open angle glaucoma in the subject.

19. The method of claim 1, wherein the patient is diagnosed with sleep apnea after being diagnosed with open angle glaucoma.

20. The method of claim 1, wherein the patient is diagnosed with vascular dysregulation and/or Flammer syndrome after being diagnosed with open angle glaucoma.
